(19) United States
(12) Patent Application Publication
(10) Pub. No.: US 2016/0235932 A1
(43) Pub. Date: Aug. 18, 2016

(54) Disposable and Sterile Venilator

(71) Applicant: Scott Rankin, Nashville, TN (US)

(72) Inventor: Scott Rankin, Nashville, TN (US)

(21) Appl. No.: 15/004,502

(22) Filed: Jan. 22, 2016

Related U.S. Application Data
(60) Provisional application No. 62/106,946, filed on Jan. 23, 2015.

(51) Int. Cl. A61M 16/00; A61M 16/04; A61M 16/22; A61M 16/08; A61M 16/20

(52) U.S. Cl. CPC A61M 16/0075; A61M 16/0875; A61M 16/208; A61M 16/0808; A61M 16/0078; A61M 16/22; A61M 16/0465; A61M 16/0003; A61M 2205/11; A61M 2205/50

(57) ABSTRACT

A sterile and disposable ventilator is described in which all of the surfaces of the air-contacting component that contact patient airflow are sterile and disposable. A method of reducing or preventing ventilator cross contamination between a first patient and a second patient is also described that uses a sterile and disposable ventilator. The air-contacting component may be constructed in one-piece component and configured to operate with a mechanical ventilator component. The sterile and disposable ventilator of the present disclosure allows for efficient and cost-effective installation of a sterile air-contacting component that may be replaced between patients. The sterile and disposable ventilator reduces or eliminates ventilator inter-patient bacterial cross-contamination, and hospital-acquired infection rates may be resultantly lowered. Because current expensive bacterial air filters and other complex maintenance programs would be eliminated, the invention(s) of this disclosure also would simplify patient care and reduce cost.

### CROSS-REFERENCE TO RELATED APPLICATIONS

This application claims priority to U.S. Provisional Patent Application No. 62/106,946, filed Jan. 23, 2015, by J. Scott Rankin, and is entitled in whole or in part to that filing date for priority. The specification of Provisional Patent Application No. 62/106,946 is incorporated herein in its entirety by reference.

### FIELD OF DISCLOSURE

The present disclosure relates generally to medical ventilators. More particularly, the disclosure pertains to a disposable and sterile full-service ventilator that includes integrated components that come into contact with patient airflow. The components that contact patient airflow may be one-piece, sterile, and disposable.

### BACKGROUND OF THE DISCLOSURE

A ventilator forces air through tubing and into a cuffed endotracheal tube positioned in a patient's trachea. There are two primary types of ventilators: volume-cycled and pressure-cycled.

In a volume ventilator, a mechanical bellows or piston is pushed with a motor to provide inspiratory air volume, and as a patient expires through a separate valved circuit, the bellows takes-in air mixed with oxygen or other gases, provided through an intake port. In current practice, repeated use of a system can be associated with condensation of water vapor contained within the system. Water vapor contained within the system may condense, providing a medium for growth of bacteria, including gram-negative bacteria. Even contamination by a small number of bacteria may result in bacterial colonization within the ventilator interior and bellows, putting future patients receiving treatment with the ventilator at risk for infection.

Pressure-cycled ventilators use gas pressure to inflate a patient's lungs and contain surfaces that may potentially be contaminated and transmit bacteria to other patients. Infection via ventilator contamination is a public health concern, the prevention of which is of large importance to the medical community. The disposable and sterile ventilator of the present disclosure may prevent or reduce the instances of cross-contamination more effectively and efficiency than previous efforts.

Lung infection after heart surgery is a major, if not the most dire, problem in current clinical practice, especially when nosocomial, i.e. hospital-acquired, organisms are involved. (See Rankin et al., “Determinants of operative mortality in valvular heart surgery”, The Journal of Thoracic and Cardiovascular Surgery, Vol. 131, Issue 3, March 2006, Pages 547-557). Evidence exists in the United States that instances of postoperative nosocomial lung infections have recently increased, and lung dysfunction is now the most common major complication after coronary bypass surgery, occurring in up to 10% of U.S. patients. Nosocomial pneumonia is primarily caused by gram-negative bacteria. Gram-negative bacteria thrive in moist environments, such the environment that typically exists inside of a ventilator. These bacteria are increasingly becoming resistant to available antibiotics, and with limited therapeutic options, mortality risk associated with postoperative pneumonia increases many fold (from 2% to 15% for valve surgery). Thus, reducing or preventing transmission of ventilator-associated bacteria would improve clinical results and reduce healthcare costs.

Indeed, a recent increase in pulmonary deaths and complications suggest that prevention and improved management of pulmonary and infectious complications is an important focus for quality improvement. Moreover, in “The STS Mitral Valve Repair/Replacement plus CABG Composite Score: A report of the STS Quality Measurement Task Force” by Rankin et al., the investigators found that in 26,463 patients who had mitral valve and coronary bypass procedures, 26% of patients experienced pulmonary morbidities, and when pulmonary morbidities occurred, post-operative mortality increased from 2% without complications to 17% with pulmonary complications. Thus, pulmonary complications comprise the major problem faced today in cardiac surgery, as well as all of in-patent medicine. To a significant extent, the problem of pulmonary morbidities is a major factor in the currently expanding number of patients in hospitals' critical and intensive care units in the US.

Before the introduction of penicillin, pneumonia was the leading cause of death in the US. After coronary artery bypass grafting (CABG) surgery, pneumonia rates varied across US hospitals from 0.6% to 6.1%. (See Likosky et al., “Sources of Variation in Hospital-Level Infection Rates After Coronary Artery Bypass Grafting: An Analysis of The Society of Thoracic Surgeons Adult Heart Surgery Database”, The Annals of Thoracic Surgery, Vol. 100, Issue 5, November 2015, Pages 1570-1576). Of the 0.6% to 6.1% range, Likosky et al. found that only 2% of the variation could be attributed to patient risk factors. (Likosky, Rankin, et al. Determinants of Hospital Variation in Pneumonia Rates after Coronary Artery Bypass Grafting: an Analysis from the Society of Thoracic Surgeons Adult Cardiac Surgery Database, submitted for publication). Thus, individual hospital's processes of care seem to be responsible and especially inadequate infection control in ventilator management in these hospitals.

Also contributing to the problem of postoperative pneumonia is the continued emergence of antibiotic resistant bacteria. Documented patient mortality has resulted from fatal multi-antibiotic-resistant gram-negative pneumonia in postoperative cardiac patients, such as in patients who have undergone cardiac valve replacement and/or coronary artery bypass surgeries. Not only are the patient fatalities due to post-operative pneumonia infection tragic, but these patients also require costly hospitalizations and consume large amounts of hospital resources. In fact, several studies of heart surgery costs have shown that the 5-10% of the patients that develop pulmonary complications consume 30-40% of total service resources. Of concern, antibiotic resistant bacteria are becoming increasing more common in these types of infections.

Hospital acquired, gram negative bacteria are sometimes referred to as “SPACE” bacteria because they most frequently comprise Serratia Marcescens, Stenotrophomonas M., Pseudomonas Aeruginosa, Acinetobacter Baumannii, Citrobacter Freundii, Enterobacter Cloacae, and their relatives such as Klebsiella, E. Coli, etc. SPACE bacteria share common characteristics, such as being gram negative rods, causing nosocomial pneumonia, having the ability to live in aqueous hospital environments, having multi-drug resistance, having the ability to interbreed, having the ability to mutate, causing endotoxic multi-organ failure in a host, and causing opportunistic infections.

Moreover, in addressing the opportunistic component of SPACE bacteria after surgery, especially heart surgery, outcomes can be positively affected by treatments such as intravenous immunoglobulin G (IV-IgG) therapy (Rankin et al, J Thorac Cardiovasc Surg 2011;142:575-580). However, intravenous immunoglobulin treatments are very costly and only seek to treat the infection rather than prevent it. Thus, a need exists for preventing postoperative infections, particularly postoperative pneumonia caused by SPACE bacteria, in patients recovering from surgery.

Without being bound to any particularly theory, Applicant believes that the leading cause of nosocomial pneumonia is patient-cross contamination between ventilators and operating room anesthesia machine respirators that are inadequately cleaned between patients, especially in light of the aqueous environment favorable for SPACE bacteria growth due to condensation within respirators and ventilators. Furthermore, technological advances with respect to respirators and ventilators have rendered them virtually impossible to fully sterilize, as respirators and ventilators have many sensitive and complicated parts.

Prior known solutions for preventing or reducing the transmission of ventilator-associated bacteria are inadequate. The problem of patent-to-ventilator and ventilator-to-patient bacteria transmission has been addressed in Europe by government mandated, complex ventilator maintenance programs. For example, the German healthcare system requires such a maintenance program. When government mandated complex ventilator maintenance programs are rigorously followed, as required by the German healthcare system, postoperative pneumonia rates may be very low, usually less than about 1% (Mazzitelli et al. Euro J Cardiothorac Surg, in press, doi:10.1093/ejcts/ezv234). However, following these complex maintenance programs is burdensome, time consuming, and expensive.

Previously attempted solutions to the problem of ventilator-associated bacteria transmission have included filtering air that passes through the connecting tubing in order to prevent bacteria originating in the ventilator from passing to the patient, or vice-versa. However, filters are not completely effective, as they may allow some particles to pass, and thus, still may allow for bacterial transmission from patient to ventilator and vice versa. Moreover, ventilator filters can be costly to frequently replace.

Another inadequate known solution has been to provide ventilator components that may be autoclaved periodically, thereby minimizing bacteria buildup. While effective in reducing postoperative lung infection from ventilators, the process of autoclaving all air-contacting components of a ventilator is impractical. Disadvantages of autoclaving include ventilator equipment downtime, extensive labor time, and high costs.

It is also known that some ventilator components, such as flow sensors, may be disassembled and cleaned by immersion in disinfectant solutions. However, cleaning agents may damage or reduce the functional life of ventilator components and involves unacceptable ventilator equipment downtime.

Previous solutions also teach only some of ventilator air-contacting components as disposable and sterile. However, these measures do not teach a completely disposable ventilator, with all air-contacting components being sterile, disposable, and replaceable. Indeed, references exist for individual components of ventilators that are sterile and disposable, but none comprise a complete, one-piece disposable and sterile component for all ventilator air-contacting surfaces.

Moreover, known solutions may require complex clinical organization, including training and the establishment and enforcement of elaborate clinical procedures. None of the known solutions include a completely disposable ventilator with all air-contacting components that are sterile and replaceable before each patient use. Because of cross-contamination, replacing only one or some of the ventilator components is ineffective if other adjoining regions within the ventilator are left un-serviced. If all of the ventilator air-contacting surfaces are not sterile, bacteria in the unsterile areas may quickly contaminate the air stream, and thus, contaminate the entire system.

Accordingly, the ventilator and methods of the present disclosure solve the need that exists for a ventilator or respirator that prevents, or reduces the instances of, transmitting ventilator-associated bacteria, particularly those that cause nosocomial lung infections, efficiently, quickly and cost-effectively. Advantageously, the ventilator of the present disclosure may be sterile, completely disposable, inexpensive, and replaced efficiently. Moreover, the ventilator of the present disclosure may have multiple configurations so that it can be used in operating rooms and intensive care units.

### BRIEF SUMMARY OF THE DISCLOSURE

In an embodiment, a ventilator comprises 1) a mechanical ventilator component including a motor and 2) a sterile, disposable, and integrated air-contacting component. The air-contacting component includes a bellows communicating with the motor and at least one patient connecting tube in gaseous communication with the bellows. The at least one tube is in gaseous communication with each of a ventilation bag, at least one valve, a carbon dioxide scrubber, and a gas inflow line. In one embodiment, the air-contacting component is constructed as or formed as one-piece. The air-contacting component may be constructed of, for example, polyurethane, low-density polyethylene, polyvinyl chloride, silicone, neoprene, polyisoprene, polyamide, polyethylene terephthalate or a combination thereof. The sterile, air-contacting component may be disposed and sealed within an interior sterile volume of a sealed vessel.

In an embodiment, the ventilator further comprises at least one sensor interface in gaseous communication with the air-contacting component. In another embodiment, the ventilator further comprises at least one central processing unit configured to control the motor. In an embodiment, the ventilator includes a water trap in gaseous communication with the at least one tube. The ventilator may further comprise a gas disposal line in gaseous communication with the at least one tube.

In another embodiment, the ventilator includes a bellows, a ventilation bag, a gas inflow cap connector, at least one valve and a carbon dioxide scrubber each in gaseous communication with the at least one tube.

In another embodiment, a disposable ventilator includes a one-piece and disposable air-contacting component in which all air-contacting surfaces are sterile.

In an embodiment, a method of reducing or preventing ventilator cross contamination between a first patient and a second patient includes providing a first disposable ventilator including at least one sterile air-contacting surface having at least one patient connecting tube; connecting the first patient to the first disposable ventilator with the at least one patient connecting tube so that the at least one first patient lung is in gaseous communication with the at least one sterile air-contacting surface; disconnecting the first patient from the first disposable ventilator; providing a second disposable ventilator including at least one sterile air-contacting surface having at least one patient connecting tube, wherein the ventilator includes at least one patient connecting tube; and connecting the second patient to the at least one patient connecting tube of the second disposable ventilator so that the at least one second patient lung is in gaseous communication with the at least one sterile air-contacting surface of the second disposable ventilator.

In an embodiment, all air-contacting surfaces of the first disposable ventilator are sterile prior to the connecting the first patient to the first disposable ventilator, and all air-contacting surfaces of the second disposable ventilator are sterile prior to the connecting the second patient to the second disposable ventilator. All air-contacting surfaces may be sterile prior to each of the connecting steps. The first disposable ventilator may be discarded after the connecting of the first disposable ventilator to the first patient.

In some embodiments, the cross contamination is the transmission of a bacteria selected from the group consisting of Serratia marcescens, Stenotrophomonas maltophilia, Pseudomonas aeruginosa, Acinetobacter baumannii, Citrobacter freundii, Enterobacter cloacae, klebsiella pneumonia, Escherichia coli and combinations thereof. In an embodiment, the reduction or prevention of cross contamination reduces instances of or prevents nosocomial lung infections in a postoperative subject.

### BRIEF DESCRIPTION OF THE DRAWINGS

FIG. 1 is a schematic view of an embodiment of the disposable and sterile ventilator.

FIG. 2 is a schematic view of another embodiment of the ventilator communicating with a mechanical ventilator.

FIG. 3 is a cross-sectional view of an embodiment of an air-contacting component disposed within a sterile sealed vessel.

FIG. 4 is a schematic view of an embodiment of the disposable and sterile ventilator.

FIG. 5 is a schematic view of an embodiment of a gas inflow source and a gas mixer cap.

FIG. 6 is a schematic view of an embodiment of a sensor and a patient tube.

### DETAILED DESCRIPTION OF THE DISCLOSURE

While the making and using of various embodiments of the present invention are discussed in detail below, it should be appreciated that the present invention provides many applicable inventive concepts that is embodied in a wide variety of specific contexts. The specific embodiments discussed herein are merely illustrative of specific ways to make and use the invention and do not delimit the scope of the invention.

To facilitate the understanding of the embodiments described herein, a number of terms are defined below. The terms defined herein have meanings as commonly understood by a person of ordinary skill in the areas relevant to the present invention. Terms such as “a,” “an,” and “the” are not intended to refer to only a singular entity, but rather include the general class of which a specific example may be used for illustration. The terminology herein is used to describe specific embodiments of the invention, but their usage does not delimit the invention, except as set forth in the claims.

As described herein, an upright position is considered to be the position of apparatus components while in proper operation or in a natural resting position as described herein. Vertical, horizontal, above, below, side, top, bottom and other orientation terms are described with respect to this upright position during operation unless otherwise specified. The term “when” is used to specify orientation for relative positions of components, not as a temporal limitation of the claims or apparatus described and claimed herein unless otherwise specified. The term “lateral” denotes a side to side direction when facing the “front” of an object.

The phrase “in one embodiment,” as used herein does not necessarily refer to the same embodiment, although it may. Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements, and/or states are in any way required for one or more embodiments or that one or more embodiments necessarily include logic for deciding, with or without author input or prompting, whether these features, elements and/or states are included or are to be performed in any particular embodiment.

The term “sterile,” as used herein means free from living microorganisms, including viruses such that the sterile object is incapable of transmitting infectious organisms, but also non-infectious materials and contaminants, such as residues, endotoxins, or toxic chemicals capable of causing patient injury. In an embodiment, the objects of the disclosure that are referred to as “sterile” are instead “aseptic,” meaning free from disease-causing contaminants such as such as bacteria, viruses, fungi and parasites. In an embodiment, an object that is “sterile” has a sterility assurance level, or a chance of non-sterile object of the number of objects, of at least 10⁻⁶. An object that has a sterility assurance level of at least 10⁻⁶ no greater than a 1 in 1,000,000 chance of being a non-sterile object.

The phrase “in gaseous communication” means that the objects in gaseous communication are configured so that gas, such as oxygen, nitrogen, carbon dioxide, and/or anesthetic gases may flow between them.

The term “cross contamination” refers to the process by which bacteria, contaminates or viruses are unintentionally transferred from one substance or object to another, particularly human patients, with harmful effect.

The terms “ventilator” and “respirator” are used interchangeably herein.

A “postoperative” subject or patient is one who recently had an operation or surgery. In an embodiment, a postoperative patient recently had cardiac surgery, such as coronary artery bypass grafting (CABG), transmyocardial laser revascularization (TMR), heart valve repair or replacement, arrhythmia treatment, an aneurysm repair, a heart replacement, any combination thereof, and/or any major operation requiring mechanical ventilation. In some embodiments, a postoperative patent had an operation or surgery within 60 days, preferably 30 days, more preferably 14 days, most preferably 2 days.

This written description uses examples to disclose the invention and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It will be understood that the particular embodiments described herein are shown by way of illustration and not as limitations of the invention. The principal features of this invention may be employed in various embodiments without departing from the scope of the invention. Those of ordinary skill in the art will recognize numerous equivalents to the specific procedures described herein. Such equivalents are considered to be within the scope of this invention and are covered by the claims.

All of the apparatuses and/or methods disclosed and claimed herein may be made and/or executed without undue experimentation in light of the present disclosure. While the apparatuses and methods of this invention have been described in terms of the embodiments included herein, it will be apparent to those of ordinary skill in the art that variations may be applied to the apparatuses and/or methods and in the steps or in the sequence of steps of the method described herein without departing from the concept, spirit, and scope of the invention. All such similar substitutes and modifications apparent to those skilled in the art are deemed to be within the spirit, scope, and concept of the invention as defined by the appended claims.

Numerous other objects, advantages, and features of the present invention will be readily apparent to those of skill in the art upon a review of the drawings and description of a preferred embodiment.

All patents and publications described or discussed herein are hereby incorporated by reference in their entirety.

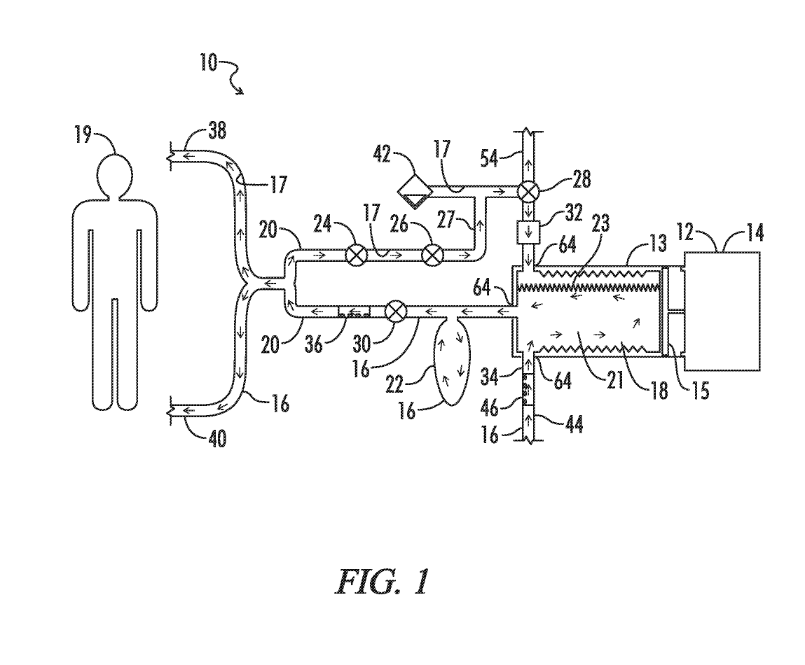

As shown in FIG. 1, a ventilator 10 may include a mechanical ventilator 12 and an air-contacting component 16. Mechanical ventilator 12 may be set to deliver a constant volume (volume cycled), a constant pressure (pressure cycled), or a combination of both with each breath of a patient 19. Modes of ventilation that maintain a minimum respiratory rate regardless of whether or not the patient initiates a spontaneous breath are referred to as assist-control (A/C). Because pressures and volumes are directly linked by the pressure-volume curve, any given volume will correspond to a specific pressure, and vice versa, regardless of whether mechanical ventilator 12 is pressure or volume cycled. Adjustable ventilator settings differ with mode but include respiratory rate, tidal volume, trigger sensitivity, flow rate, waveform, and inspiratory/expiratory (I/E) ratio.

Mechanical ventilator 12 may include a piston chamber 13 in which a piston or compressor 15 is in contact with an air-contacting component 16 that is disposed. Air-contacting component 16 may include a bellows 18 disposed within piston chamber 13. Piston 15 may be oscillatedly driven by an electric motor 14 to compress gas in bellows 18, which creates air flow within the air-contacting component 16 to move air to and from at least one lung of patient 19. Motor 14 causes force to compresses gas within bellows 18, raising the pressure within it, which causes gas to flow into at least one lung of patient 19. The dimensions of bellows 18, and thereby a bellows volume 21, may be varied depending on the needs of patient 19. Bellows 18 may have bellows volume 21 of between 10 mL and 2 L, preferably between 300 mL and 1.5 L. Mechanical ventilator 12 may be configured to deliver, for example, between about 0.15 L/min and 20 L/min of gas to patient 19, configured to have an inspiratory-to-expiratory time ratio (I:E) of between about 1:1-1:5, and configured to have a ventilatory frequency of 8-150 breaths per minute.

Air-contacting component 16 may friction fit with or be directly attached to mechanical ventilator 12. In another embodiment, air-contacting component 16 is configured to be in gaseous communication with a pressure-driven ventilator (not shown). Specifically, bellows 18 may friction fit with mechanical ventilator 12 in piston chamber 13. Bellows 18 may be configured to be operable within piston chamber 13 such that movement of piston 15 expands and contracts the bellows. Piston chamber 13 may include at least one aperture 64 through which air-contacting component 16 passes. Disposable and sterile ventilator 10 may be interoperable with existing mechanical ventilators 12 or be configured to be operable with a specially designed mechanical ventilator 12.

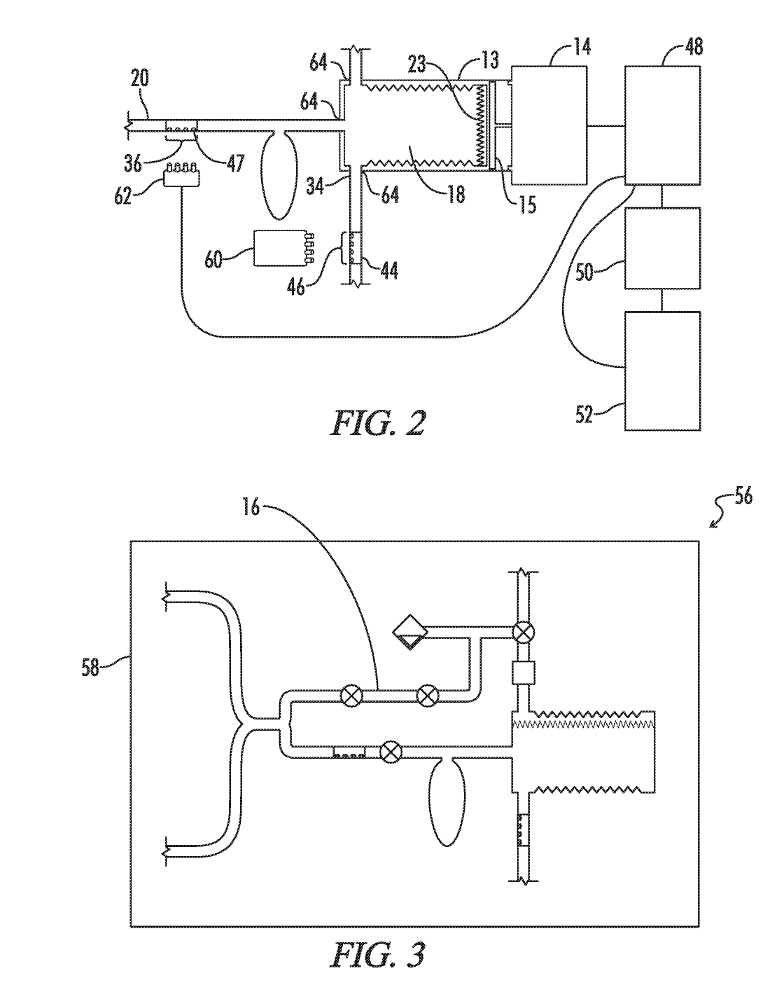

Bellows 18 may include at least one spring 23 to maintain expansion and communication of bellows 18 with piston 15. At least one spring 23 may extend across the length of bellows 18 (shown in FIG. 3) and across the height of bellows 18 (shown in FIG. 2). Bellows volume 21 may relate proportionally to the size and components of mechanical ventilator 12, particularly piston chamber 13.

Air-contacting component 16 includes at least one air-contacting surface 17. At least one air-contacting surface 17 is an interior surface of the air-contacting component, such as an interior surface of bellows 18. In an embodiment, all air-contacting surfaces are sterile prior to use. To ensure sterility, air-contacting component may be sealed in, for example, a vessel 56 (shown in FIG. 3). Vessel 56 may be a rigid container constructed of a polymer, glass or a metal. In another embodiment, vessel 56 may be flexible and sealed by heat sealing or vacuum sealing. Sterile air-contacting component 16 may be disposed within a sterile interior volume 58 of vessel 56.

Air-contacting component 16 may include at least one patient connecting tube 20 in gaseous communication with bellows 18. Patient connecting tube 20 may lead to or from patient 19 and carry gas to or from patient 19. Patient connecting tube 20 may further comprise a tracheal tube 38 and/or an endotracheal tube 40. Tracheal tube 38 connects with patient 19 via a surgical opening in the trachea of patient. Endotracheal tube 40, on the other hand, may interface with the patient by being inserted through the mouth (orotracheal) or nose (nasotracheal) into the trachea of patient 19. Tracheal tube 38 and/or endotracheal tube 40 facilitate respiration, or the exchange of oxygen and carbon dioxide, in the at least one lung of patient 19. Endotracheal tube 40 may drain liquid excretions from patient 19.

Air-contacting component 16 may further comprise a hand ventilation bag or reservoir bag 22 in gaseous communication with patient connecting tube 20. Bag 22 may inflate and deflate with piston 15 movement such that bag 22 represents the at least one lung of patient 19, signaling to health care providers the functionality of ventilator 10. Bag 22 may also replace the function of mechanical ventilator 12 in such instances as an emergency failure of mechanical ventilator 12 in that a health care provider can pump bag 22 to manually move gas through air-contacting component 16 and to and from patient 19.

In an embodiment, air-contacting component 16 comprises a positive end-expiratory pressure (PEEP) valve 24. PEEP valve 24 may be disposed between a gas disposal line 54 and patient 19 and in gaseous communication with patient connecting tube 20. PEEP valve 24 establishes positive end-expiratory pressure, or the maintenance of positive pressure within the lungs, at the end of expiration. Air-contacting component 16 may also include an expiratory valve 26. Expiratory valve 26 may be disposed between PEEP valve 24 and gas disposal line 54. In some embodiments, air-contacting component 16 includes an exhalation circuit 27. Expiratory valve 26 may regulate gas flow away from the patient and be in gaseous communication with exhalation circuit 27. Exhalation circuit 27 may include a water trap 42 and a gas disposal line 54 in gaseous connection with a hospital waste gas collection system (not shown). Water trap 42 may be configured to collect condensation from the air within air-collecting component 16.

Air-contacting component 16 may comprise a closed circuit rebreathing valve 28 disposed between the bellows 18 and expiratory valve 26 and in gaseous communication with patient connecting tube 20. Rebreathing valve 26 may be a one-way valve that allows air to travel from patient 19 to bellows 18 when open, partially restricts air from patient 19 to bellows 18 when partially closed and does not allow air to pass from patient 19 to bellows 18 when closed. Rebreathing valve 26 may, for example, prevent ambient air from lowering oxygen concentration during spontaneous breathing when partially or completely closed.

In one embodiment, air-contacting component 16 comprises an inspiratory valve 30 disposed between bellows 18 and patient 19 and is in gaseous communication with patient connecting tube 20. Inspiratory valve 30 may control gas flow to patient 19 from bellows 18. Inspiratory valve 30 opens during the inspiratory phase until the respiratory gas of the respective breath is completely delivered. During the entire mechanical inspiratory time, the expiration valve is completely closed during controlled ventilation. Expiration valve 30 opens at the beginning of the expiratory phase and releases the respiratory gas. The timings and durations of the Inspiratory and expiratory phases may be controlled by a central processing unit or mechanical control 48 (FIG. 2). Valves 26, 28 and 30 may be also sterile and disposable, and configured for one-way gas flow from bellows 18 to patient 19 and from patient 19 to exhalation circuit 27. In the embodiment shown in FIG. 1, the illustrative arrows in the air-contacting component indicate air-flow.

In an embodiment, air-contacting component 16 includes a sterile-disposable carbon dioxide scrubber 32. Carbon dioxide scrubber 32 may be in gaseous communication with patient connecting tube 20 and bellows 18. Carbon dioxide scrubber 32 may treat exhaled gas from patient 19, particularly when rebreathing valve 28 is partially or completely open. Carbon dioxide scrubber 32 may comprise a mineral that binds carbon dioxide, such as calcium oxide, serpentinite, a magnesium silicate hydroxide or olivine, soda lime, a molecular sieve or a combination thereof. Gas may flow from patient 19 through carbon dioxide scrubber 32 and to bellows 18.

As shown in the embodiment in FIG. 2, air-contacting component 16 includes a gas inflow line 34 in gaseous communication with bellows 18 to provide gas to bellows 18. Gas inflow line 34 may provide air-contacting component 16 with gas from a gas inflow source 60. In some embodiments, an anesthetic is supplied to the gas inflow line 34. Supplied gases may include oxygen, water vapor, ambient air, nitrous oxide, sevoflurane, desflurane, isoflurane or a combination thereof. Gas inflow source 60 and gas mixer cap may be in gaseous communication via a gas inflow interface 46. Gas inflow interface 46 may comprise one or more ports configured to receive gas inflow source 60.

Air-contacting component 16 may include at least one sensor interface 36 configured to receive one or more sensors 62. Sensor interface 36 may be disposed, for example, on patient connecting tube 20. The air-tight sterile-disposable sensor interface 36 is in continuity with the disposable tubing and may include one or more ports 47 configured to receive sensor 62. Sensors 62 may, for example, sense data such as the levels of oxygen saturation, carbon dioxide concentration, anesthesia concentration, airway pressure and volume air flow. Data sensed and gathered by sensors 62 may be transmitted to central processing unit or mechanical control 48. Central processing unit or mechanical control 48 may adjust, for example, levels of oxygen saturation, carbon dioxide concentration, anesthesia concentration, airway pressure and volume air flow by modulating valves 24, 28 and 30 and modulating piston 15 movement and timing. Central processing unit or mechanical control 48 may output data, such as the sensed data to a display 52. Medical personnel may monitor, for example, the sensed data displayed on display 52. In one embodiment, medical personnel can manually adjust via the air-tight sterile-disposable gas inflow interface, for example, levels of oxygen saturation, carbon dioxide concentration, anesthesia concentration, airway pressure volume air flow and piston movement by a user input device 50, such as a keyboard or dials, that communicates with the central processing unit or mechanical control. Central processing unit or mechanical control unit 50 may control the type of ventilation, for example, closed circuit, gas supply or anesthesia functions, and modulate to maintain stored presets of these levels.

Sterile air-contacting component 16 may be constructed as one-piece. The one-piece construction is advantageous for maintaining sterility and ease of installing during use by medical personnel. Another benefit of the one-piece construction is enablement of quick and cost-effective installation of different sterile air-contacting surfaces for the entire ventilator before use in different patients. Installing the sterile, disposable component 16 within a ventilator, regardless of whether mechanical or pressure driven, may take a minimal amount of time, for example, less than five minutes. With this unique approach, inter-patient bacterial cross-contamination would be eliminated, and hospital-acquired infection rates lowered. Because current expensive bacterial air filters and other complex maintenance programs will be eliminated, this invention may also simplify patient care and reduce cost.

All air-contacting surfaces 17 may be air-tight, sterile, and in gaseous communication with one another. Sterile air-contacting component 16 may be constructed of, for example, polyurethane, low-density polyethylene, polyvinyl chloride, silicone, neoprene, polyisoprene, polyamide, polyethylene terephthalate or a combination thereof.

Table 1, shown below, summarizes components in one embodiment of ventilator 10, their functions, and whether they are part of air-contacting component 16 or mechanical ventilator 12.

TABLE 1
Example Components of One Embodiment of Ventilator

| Component | Function | Ventilator Section |
|---|---|---|
| Patient Connecting Tube | Connects Endotracheal Tube to Bellows | Sterile Air-Contacting |
| Tracheal Tube | Closed Tubing for Aspiration of Endotracheal | Sterile Air-Contacting |
| | Tube | |
| Hand Ventilation Bag | Allows Manual Ventilation of Air Volume | Sterile Air-Contacting |
| Bellows | Pushes Pre-determined Air Volume into Lungs | Sterile Air-Contacting |
| Gas Mixer Cap | Attaches to Gas Inflow Source | Sterile Air-Contacting |
| Sensor Interface | Attaches to Sensors | Sterile Air-Contacting |
| Insp./Exp. Valves | Maintains Unidirectional Ventilation | Sterile Air-Contacting |
| PEEP Valve | Establishes Positive End-Expiratory Pressure | Sterile Air-Contacting |
| Water Trap | Collects Water Vapor Condensation in Airway | Sterile Air-Contacting |
| CO² Canister | Absorbs and Removes CO₂ from the Air Flow | Sterile Air-Contacting |
| Gas Disposal Line | Directs Used Anesthesia Gases for Disposal | Sterile Air-Contacting |
| Piston | Expands and Contracts Bellows | Mechanical |
| Gas Inflow Source | Mixes % of Various Gases; Receives/Distributes | Mechanical |
| | Volatile Anesthesia Gases | |
| Sensors | Sense Pressure, Flow, O₂, CO₂, Volatile Gas | Mechanical |
| Computer Processing Unit | Regulates Movement of Piston | Mechanical |
| Display | Visually Transmits Ventilator Data to User | Mechanical |

Gas inflow source 60 and sensor 62 may be cleaned periodically, for example, by disinfectants or autoclaving. Gas inflow source 60 and sensor 62 may be constructed of metal or a polymer, preferably metal, and may be cleaned or autoclaved.

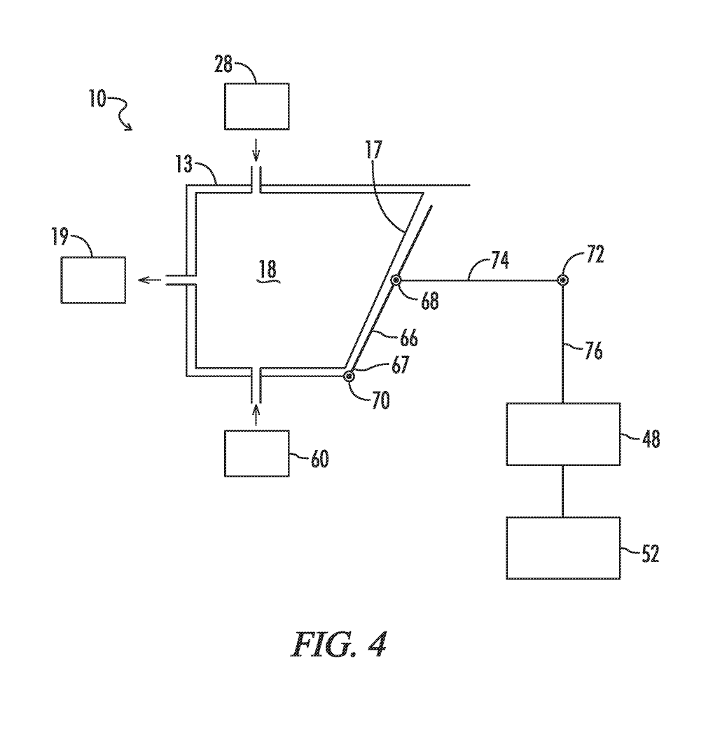

In FIG. 4, an embodiment of the sterile and disposable ventilator includes a bellows chamber 13 in which a bellows 18 is disposed. Bellows 18 may be compressed and expanded by movement of pusher plate 66. Pusher plate is configured to pivot around chamber hinge 70. Chamber hinge 70 may connect chamber 13 to pusher plate 66. Chamber hinge 70 may be disposed on an end 67 of pusher plate 66. Pusher plate 66 may connect to a pusher plate arm 74 at plate hinge 68. Pusher plate may pivot at plate hinge 68. Pusher plate arm 74 and pusher plate 66 may be configured such that lateral movement of pusher plate arm 74 pivots pusher plate 66, forcing gas in and out of bellows 18. Gas may flow into bellows 18 from a gas inflow source 60 and from rebreathing air passing through open or partially open rebreathing valve 28 to patient 19. Pusher plate arm 74 may be hingedly connected to a driving arm 76 at arm hinge 72. Plate hinge 68 and arm hinge 72 may comprise a mechanical linkage, such as cam assembly, to transfer movement from one direction to another direction. For example, arm hinge 72 may comprise a cam assembly transfer vertical movement of driving arm 76 to lateral movement of plate arm 74. Driving arm 76 may be configured to communicate with a central processing unit or mechanical control 48. Central processing unit or mechanical control 48 may communicate with a display 52.

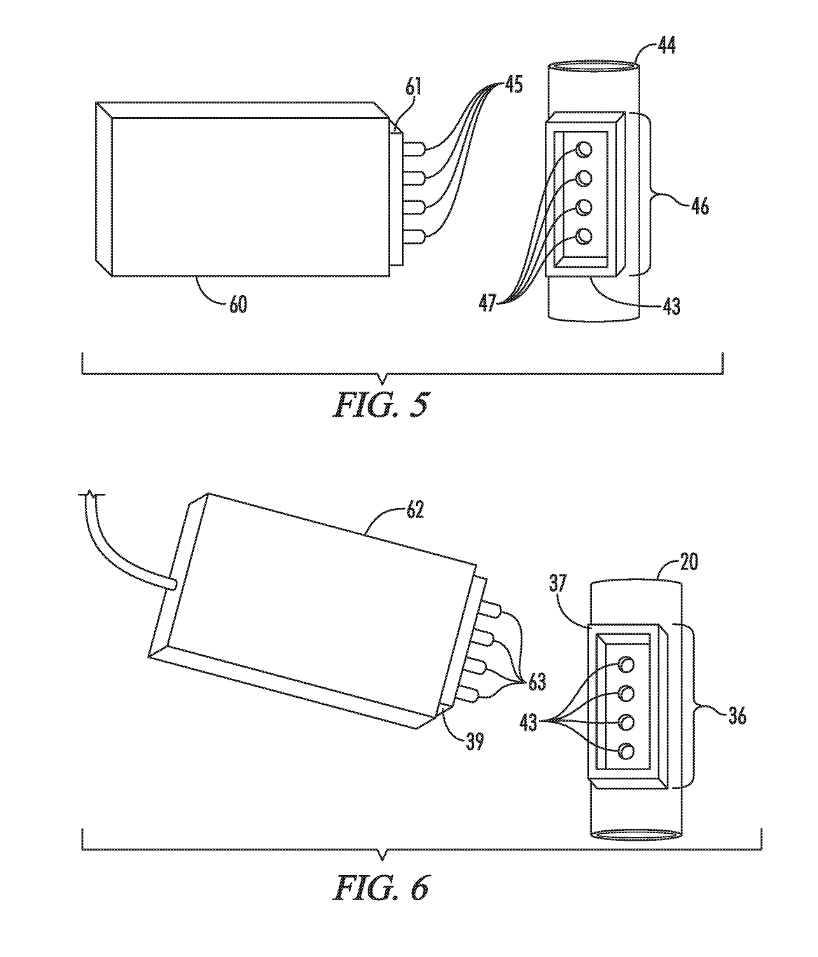

FIG. 5 shows an embodiment of gas mixer cap 44 and gas inflow source 60. Gas mixer cap 44 may comprise a gas inflow interface 46. Gas flow interface 46 may be integrally formed with gas mixer cap 44. Gas inflow interface 46 may include a sidewall 43 around at least one port 47. Port 47 may be configured to be in gaseous communication with air contacting surface 17 of air contacting component 16. Sidewall 43 may be configured to receive a raised portion 61 of gas flow source 60 such that raised portion 61 and sidewall 43 friction fit, forming a seal. Port 47 may engage with at least one outlet 45 on gas inflow source 60. Port 47 may sealingly engage or friction fit outlet 45. Port 47 may be configured to receive gas from gas inflow source 60 and communicate gas to gas mixer cap 44.

FIG. 6 shows an embodiment of sensor 62 and sensor interface 36. Sensor interface 36 may be disposed on at least one patient connecting tube 20. Sensor interface 36 may be integrally formed with connecting tube 20. Sensor interface may have a sensor wall 37 around at least one nozzle 43. Nozzle 43 may be configured to be in gaseous communication with air contacting surface 17 of air contacting component 16. Sensor wall 37 may be configured to receive a raised portion 39 of sensor 62 such that raised portion 39 and wall 37 friction fit, forming a seal. Nozzle 43 may engage with at least one sensor channel 63 on sensor 62. Nozzle 43 may sealingly engage or friction fit channel 63. Nozzle 43 may be configured to transmit gas from and to patient connecting tube 20 to and from sensor 62.

In an embodiment, a method of reducing or preventing ventilator cross contamination between a first patient and a second patient, comprises providing a first disposable ventilator including at least one sterile air-contacting surface, wherein the ventilator includes at least one patient connecting tube; connecting the first patient to the first disposable ventilator with the at least one patient connecting tube so that an at least one lung of the patient is in gaseous communication with the at least one sterile air-contacting surface; disconnecting the first patient from the first disposable ventilator; providing a second disposable ventilator including at least one sterile air-contacting surface, wherein the ventilator includes at least one patient connecting tube; and connecting the second patient to the at least one patient connecting tube of the second disposable ventilator so that the at least one lung of the second patient is in gaseous communication with the at least one sterile air-contacting surface of the second disposable ventilator.

In an embodiment, all air-contacting surfaces of the first disposable ventilator are air-tight and sterile prior to the connecting the first patient to the first disposable ventilator, and all air-contacting surfaces of the second disposable ventilator are sterile prior to the connecting the second patient to the second disposable ventilator. All air-contacting surfaces may be sterile prior to each of the connecting steps.

In an embodiment, the first ventilator is operated after the connecting to the first patient. The operating may include expanding and contracting a bellows in gaseous communication with the at least one patient connecting tube of the first disposable ventilator. The ventilator may be disposable such that it is discarded after use with the first patient, such that the second patient is connected to a second disposable ventilator. In embodiments where components such as the gas inflow source, sensors, and mechanical ventilator are not disposable, they may be sterilized by autoclaving or cleaning solutions between being connected to the first ventilator and the second ventilator.

The sterile and disposable component may be changed and replaced with a new sterile component periodically for use with the same patient. Advantageously, replacing the sterile component for patient requiring ventilation for a prolonged period may avoid bacterial buildup in the ventilator. The sterile component may also be changed and replaced with a new sterile component for use in a ventilator prior to treating a different patient.

In one embodiment, the cross contamination is the transmission of a bacteria which may include Serratia marcescens, Stenotrophomonas maltophilia, Pseudomonas aeruginosa, Acinetobacter baumannii, Citrobacter freundii, Enterobacter cloacae, klebsiella pneumonia, Escherichia coli and combinations thereof. The reduction or prevention of cross contamination may reduce instances of or prevent nosocomial lung infections in a postoperative subject. In an embodiment, the cross contamination may be any bacteria, virus, or potentially pathologic organism or substance.

What is claimed is:

1. A ventilator comprising:
a mechanical ventilator including a motor;
a sterile, disposable and integrated air-contacting component including
a bellows communicating with the motor;
at least one patient connecting tube in gaseous communication with the bellows;
at least one valve on the at least one tube; and
a gas inflow line in gaseous communication with the bellows.

2. The ventilator of claim 1, wherein the sterile and disposable air-contacting component is constructed as one-piece.

3. The ventilator of claim 1, further comprising at least one sensor interface in gaseous communication with the air-contacting component.

4. The ventilator of claim 1, further comprising at least one central processing unit configured to control the motor.

5. The ventilator of claim 1, wherein the air-contacting component further comprises a water trap in gaseous communication with the at least one tube.

6. The ventilator of claim 1, wherein the air-contacting component further comprises a gas disposal line in gaseous communication with the at least one tube.

7. The ventilator of claim 1, wherein the air-contacting component is constructed of polyurethane, low-density polyethylene, polyvinyl chloride, silicone, neoprene, polyisoprene, polyamide, polyethylene terephthalate or a combination thereof.

8. The ventilator of claim 1, further comprising a sealed vessel including a sterile interior chamber, and wherein the sterile air-contacting component is disposed within the sterile interior chamber.

9. The ventilator of claim 1, further comprising a bellows, a ventilation bag, a gas inflow cap connector, at least one valve, a water trap, and a carbon dioxide scrubber each in gaseous communication with the at least one tube.

10. A disposable ventilator, comprising:
a one-piece and disposable air-contacting component in which all air-contacting surfaces are sterile.

11. The ventilator of claim 10, further comprising a sealed vessel including a sterile interior volume, and wherein the sterile air-contacting component is disposed within the sterile interior volume.

12. A method of reducing or preventing ventilator cross contamination between a first patient and a second patient, comprising:
providing a first disposable ventilator including at least one sterile air-contacting surface, wherein the ventilator includes at least one patient connecting tube;
connecting the first patient to the first disposable ventilator with the at least one patient connecting tube so that the at least one first patient lung is in gaseous communication with the at least one sterile air-contacting surface;
disconnecting the first patient from the first disposable ventilator;
providing a second disposable ventilator including at least one sterile air-contacting surface, wherein the ventilator includes at least one patient connecting tube; and
connecting the second patient to the at least one patient connecting tube of the second disposable ventilator so that the at least one second patient lung is in gaseous communication with the at least one sterile air-contacting surface of the second disposable ventilator.

13. The method of claim 12, wherein the air-contacting surface is constructed of polyurethane, low-density polyethylene, polyvinyl chloride, silicone, neoprene, polyisoprene, polyamide, polyethylene terephthalate or a combination thereof.

14. The method of claim 12, wherein all air-contacting surfaces of the first disposable ventilator are sterile prior to connecting the first patient to the first disposable ventilator, and all air-contacting surfaces of the second disposable ventilator are sterile prior to the connecting the second patient to the second disposable ventilator.

15. The method of claim 12, wherein the cross contamination is the transmission of a bacteria comprising Serratia marcescens, Stenotrophomonas maltophilia, Pseudomonas aeruginosa, Acinetobacter baumannii, Citrobacter freundii, Enterobacter cloacae, klebsiella pneumonia, Escherichia coli, combinations thereof, or any other bacteria.

16. The method of claim 12, wherein the reduction or prevention of cross contamination reduces instances of or prevents nosocomial lung infections in a postoperative subject.

17. The method of claim 12, wherein all air-contacting surfaces are sterile prior to each of the connecting steps.

18. The method of claim 12, further comprising expanding and contracting a bellows in gaseous communication with the at least one patient connecting tube of the first disposable ventilator.

19. The method of claim 12, further comprising discarding the first disposable ventilator after the connecting the first disposable ventilator to the first patient.

20. The method of claim 12, wherein a gas inflow source is in gaseous communication with the first ventilator, and wherein the gas inflow source is sterilized prior to being in gaseous communication with the second ventilator.
